(19) United States
(12) Patent Application Publication
(10) Pub. No.: US 2013/0123839 A1
(43) Pub. Date: May 16, 2013

(54) CHEMICAL KNOTS FOR SUTURES

(71) Applicants: Tim Sargeant, Hamden, CT (US); Jonathan Thomas, New Haven, CT (US); Ferass Abuzaina, Shelton, CT (US); Ahmad Robert Hadba, Fort Worth, TX (US)

(72) Inventors: Tim Sargeant, Hamden, CT (US); Jonathan Thomas, New Haven, CT (US); Ferass Abuzaina, Shelton, CT (US); Ahmad Robert Hadba, Fort Worth, TX (US)

(73) Assignee: TYCO HEALTHCARE GROUP LP, New Haven, CT (US)

(21) Appl. No.: 13/637,154

(22) PCT Filed: Mar. 24, 2011
(86) PCT No.: PCT/US11/29842

§ 371 Date: Jan. 9, 2013

Related U.S. Application Data
(60) Provisional application No. 61/317,427, filed on Mar. 25, 2010.

(51) Int. Cl. A61B 17/04

(52) U.S. Cl. CPC A61B 17/04
USPC 606/228

(57) ABSTRACT

Surgical sutures capable of forming a chemical knot include a first reactive member on a first portion thereof and a second reactive member on a second portion thereof, wherein the first and second reactive members are complimentary.

### BACKGROUND

1. Technical Field

The present disclosure relates to surgical sutures, and more particularly to surface activated surgical sutures capable of forming chemically bonded knots.

2. Background of Related Art

Surgeons are constantly seeking better and stronger knot-tying materials and methods. This is true in the fields of arthroscopy and laparoscopy, as well as in the field of open procedures. Arthroscopic and laparoscopic procedures, however, may be more technically demanding due to limited accessibility, as compared to open procedures. For example, the task of tying secure knots may prove to be more difficult during an arthroscopic procedure considering that the surgeon is required to tie the suture knot away from the defect and use a knot pusher to slide and/or tension the knot into position. Of course, whether performed arthroscopically, laparoscopically, or openly, suture knots must be securely tied and provide optimal knot security (resistance to loosening and/or slipping of the knot).

In arthroscopic procedures such as meniscal repair, the suture knots commonly consist of an initial sliding knot which is followed by a series of half-hitches to prevent slack in the slip knot. The addition of the half-hitches enhances knot security but also produces a larger knot profile. Knots having a larger profile may rub against the surrounding tissue causing pain and discomfort. In more severe situations, the larger knots may rub against the cartilage resulting in the formation of osteoarthritis. Also, larger knots place larger amounts of suture material into the body thereby increasing the likelihood of developing inflammation and/or infection at or near the site of the knot. Therefore, it would be beneficial to provide a suture capable of forming a chemical knot which displays enhanced knot security without the need to increase the knot profile.

### SUMMARY

The present disclosure describes surgical sutures capable of forming a chemical knot. The surgical sutures include at least one filament having an outer surface, a first portion of which includes at least one first reactive member and a second portion of which includes at least one second complimentary reactive member. The first and second reactive members are capable of chemically bonding the first portion of the filament to the second portion of the filament to form the chemical knot. In embodiments, the suture is a monofilament suture. In embodiments, the suture is a multifilament suture.

In embodiments, the suture may be tied into a knot prior to the formation of the chemical knot. In embodiments, the chemical knot may be used alone to sustain the suture in a knotted position. Methods of forming the sutures and the chemical knots are also described.

### BRIEF DESCRIPTION OF THE DRAWINGS

FIG. 1 A schematically illustrates a surgical suture in accordance with an embodiment described herein.

FIG. 1B schematically illustrates a surgical suture in accordance with an embodiment described herein.

FIG. 1C schematically illustrates a surgical suture in accordance with an embodiment described herein.

FIG. 1D schematically illustrates a surgical suture in accordance with an embodiment described herein.

FIG. 1E schematically illustrates a surgical suture in accordance with an embodiment described herein.

FIG. 2 schematically illustrates the surgical suture of FIG. 1 tied in a knot in accordance with an embodiment described herein.

FIG. 3 schematically illustrates a surgical suture tied in a knot in accordance with an embodiment described herein.

### DETAILED DESCRIPTION

The present disclosure describes surgical sutures having at least a portion of the suture surface activated. The surface activated sutures include a first reactive member and a second complimentary reactive member. The first and second reactive members may be positioned on different portions of the suture surface. The first and second reactive members positioned on the surface of the sutures described herein are capable of forming a chemical bond when placed in close proximity to one another, such as when being tied into a suture knot. The addition of a catalyst may or may not be necessary to enhance the interaction between the first and second reactive members.

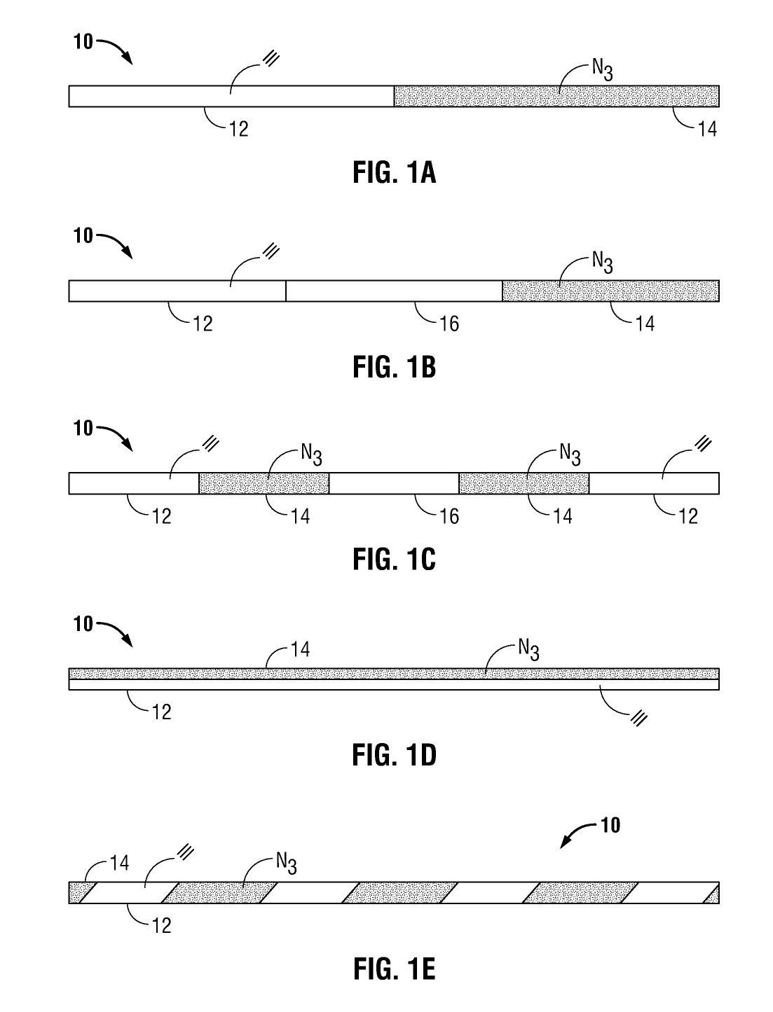

Turning now to FIGS. 1A-1E, surgical suture 10, made from a biocompatible polymeric material, is schematically shown as a single fiber or monofilament having a first portion 12 of the suture surface functionalized with a first reactive member, e.g., an alkyne group, and a second portion 14 of the suture surface functionalized with a second complimentary reactive member, e.g., an azide group. As depicted in FIGS. 1A-1E, the first reactive members are alkynes and the second reactive members as azides. Of course, the first and second reactive members are not meant to be limited to these two specific reactive members, however for purposes of clarity the azide/alkyne complimentary reactive members have been added to the figures.

In FIG. 1A, each of the first and second portions 12 and 14 represent approximately half the length of the suture. In FIG. 1B, first portion 12 is separated from second portion 14 by spacer portion 16. Spacer portion 16 is not functionalized with a first or second reactive member thereby creating space between the complimentary reactive members. Spacer portion 16 may be beneficial in preventing first and second reactive members from reacting prematurely. In embodiments, spacer portion 16 may include any biocompatible material incapable of reacting with either the first or the second reactive member.

In embodiments, the first and second portions of the suture surface may represent any design or configuration. For example in FIG. 1D, first portion 12 and second portion 14 run the entire length of the suture and create a vertical separation rather than the longitudinal separation shown in FIG. 1C. In still other embodiments, the first and second portions may be configured in a candy-cane striped manner as shown in FIG. 1E. It is further envisioned that the first and second activated portions may be positioned on the surface of the suture in any pattern or mosaic design.

The sutures can be formed from any sterilizable biocompatible material that has suitable physical properties for the intended use of the suture. The sutures described herein may be monofilaments or multifilaments sutures. The biocompatible polymers used to form the sutures may be a homopolymer or a copolymer, including random copolymer, block copolymer, or graft copolymer. The biocompatible polymer may be a linear polymer, branched polymer, or a dendrimer.

The sutures can be made from synthetic or natural polymers that are biodegradable and/or non-biodegradable. Some specific non-limiting examples of suitable biodegradable materials include polymers such as those made from carbonates (e.g., trimethylene carbonate, tetramethylene carbonate, and the like), s-caprolactone, dioxanones (e.g., 1,4-dioxanone, 1,3-dioaxanone, and the like), glycolide, lactide, dioxepanones (e.g., 1,4-dioxepan-2-one and 1,5-dioexpan-2-one), ethylene glycol, ethylene oxide, esteramides, hydroalkanoates (e.g., γ-hydroxyvalerate, β-hydroxypropionate, hydroxybuterates), poly(othroesters), tyrosine carbonates, polyimide carbonates, polyimino carbonates such as poly(bisphenol A-iminocarbonate) and poly(hydroquinone-iminocarbonate), polyurethanes, polyanhydrides, polymer drugs (e.g., polydiflunisol, polyaspirin, and protein therapeutics) and copolymers and combinations thereof. Suitable natural biodegradable polymers include collagen, cellulose, poly(amino acids), polysaccharides, hyaluronic acid, catgut, and copolymers and combinations thereof.

Examples of suitable non-degradable polymers from which the sutures described herein may be made include, but are not limited to fluorinated polymers (e.g., fluoroethylenes, propylenes, fluoroPEGs), polyolefins such as polyethylene, polypropylene, polyesters such as poly(ethylene terepththalate) (PET), nylons, polyamides, polyurethanes, silicones, ultra high molecular weight polyethylene (UHMWPE), polybutesters, polyaryletherketone, copolymers and combinations thereof It should also be understood that combinations of biodegradable and non-biodegradable materials may be used to form the sutures.

Methods for preparing materials suitable for making sutures as well as techniques for making sutures from such materials are within the purview of those skilled in the art. Some examples include, but are not meant to be limited to, extruding processes, molding processes, wet-spinning processes, gel-spinning processes and the like. In addition, the sutures may be further processed to form braids, yams and the like and in alternative embodiments, the sutures may be barbed, calendared or further processed to alter the porosity and/or the surface area of the suture.

The surgical sutures described herein are surface activated to include at least one first reactive member and at least one second complimentary reactive member. By complimentary it is meant that the first reactive member is able to interact with the second reactive member to form a chemical bond. By surface activated, the first and second reactive members are positioned near an outer surface of the suture. In this position, the first and second reactive members may be positioned in close proximity to one another when the suture is tied into a knot.

In embodiments, the first reactive group may be positioned on a first portion of the suture surface and the second complimentary reactive group may be positioned on a second portion of the suture surface. The first portion of the suture surface may come in contact with the second portion of the suture during the knot-tying process. In embodiments, the first and second reactive members will interact after the suture has been knotted and the chemical bond formed between the two members will further enhance the knot tensile strength and knot security.

In embodiments, the first and second portions of the surface of the bioabsorbable polymeric suture are functionalized with electrophilic or nucleophilic functional groups, such that, for example, a nucleophilic functional group on the first portion of the suture surface may react with an electrophilic functional group on the second portion of the suture surface to form a covalent bond.

Virtually any nucleophilic group can be used to functionalize the first portion of the suture surface, so long as a reaction can occur with the electrophilic group on the second portion of the suture surface. Analogously, virtually any electrophilic group can be used to functionalize the first portion of the suture surface, so long as a reaction can take place with the nucleophilic group on the second portion of the suture surface. In embodiments, the reaction occurs without need for catalysts, ultraviolet or other radiation. In embodiments, the reactions between the complementary members should be complete in under 60 minutes, in embodiments under 30 minutes, in yet other embodiments, the reaction occurs in about 5 to 15 minutes or less.

Non-limiting examples of nucleophilic groups include, but are not limited to, —NH₂,—NHR, —N(R)₂, —SH, —OH, —COOH, —C₆H₄—OH, —PH₂, —PHR, —P(R)₂, —NH—NH₂, —CO—NH—NH₂, C₅H₄N, etc. wherein R is hydrocarbyl, typically C₁-C₄ alkyl or monocyclic aryl. Organometallic moieties are also useful nucleophilic groups for the purposes of this disclosure, particularly those that act as carbanion donors. Examples of organometallic moieties include: Grignard functionalities —RMgHal wherein R is a carbon atom (substituted or unsubstituted), and Hal is halo, typically bromo, iodo or chloro; and lithium-containing functionalities, typically alkyllithium groups; sodium-containing functionalities.

It will be appreciated by those of ordinary skill in the art that certain nucleophilic groups must be activated with a base so as to be capable of reaction with an electrophile. For example, when there are nucleophilic sulfhydryl and hydroxyl groups on a first portion of the suture surface, the composition must be admixed with an aqueous base in order to remove a proton and provide an —S— or —O— species to enable reaction with an electrophile. Unless it is desirable for the base to participate in the reaction, a non-nucleophilic base is used. In some embodiments, the base may be present as a component of a buffer solution.

The selection of electrophilic groups provided on a second portion of the suture surface is made so that reaction is possible with the specific nucleophilic groups on the first portion of the surface of the bioabsorbable polymeric suture. Thus, when the first portion of the suture surface is functionalized with amino groups, the second portion of the suture surface may be functionalized with groups selected so as to react with amino groups. Analogously, when the first portion of the suture surface is functionalized with sulfhydryl moieties, the corresponding electrophilic groups on the second portion of the suture surface may be sulfhydryl-reactive groups, and the like.

By way of example, when a first portion of the suture surface is functionalized with amino groups (generally although not necessarily primary amino groups), the electrophilic groups present on the second portion of the suture surface are amino reactive groups such as, but not limited to: (1) carboxylic acid esters, including cyclic esters and “activated” esters; (2) acid chloride groups (—CO—Cl); (3) anhydrides (—(CO)—O—(CO)—R); (4) ketones and aldehydes, including α,β-unsaturated aldehydes and ketones such as —CH═CH—CH═O and —CH═CH—C(CH₃)═O; (5) halides; (6) isocyanate (—N═C═O); (7) isothiocyanate (—N═C═S); (8) epoxides; (9) activated hydroxyl groups (e.g., activated with conventional activating agents such as carbonyldiimidazole or sulfonyl chloride); and (10) olefins, including conjugated olefins, such as ethenesulfonyl (—SO₂CH═CH₂) and analogous functional groups, including acrylate (—CO₂—C═CH₂), methacrylate (—CO₂—C(CH₃)═CH₂)), ethyl acrylate (—CO₂—C(CH₂ CH₃)═CH₂), and ethyleneimino (—CH═CH—C═NH). Since a carboxylic acid group per se is not susceptible to reaction with a nucleophilic amine, components containing carboxylic acid groups must be activated so as to be amine-reactive. Activation may be accomplished in a variety of ways, but often involves reaction with a suitable hydroxyl-containing compound in the presence of a dehydrating agent such as dicyclohexylcarbodiimide (DCC) or dicyclohexylurea (DHU). For example, a carboxylic acid can be reacted with an alkoxy-substituted N-hydroxy-succinimide or N-hydroxysulfosuccinimide in the presence of DCC to form reactive electrophilic groups, the N-hydroxysuccinimide ester and the N-hydroxysulfosuccinimide ester, respectively. Carboxylic acids may also be activated by reaction with an acyl halide such as an acyl chloride (e.g., acetyl chloride), to provide a reactive anhydride group. In a further example, a carboxylic acid may be converted to an acid chloride group using, e.g., thionyl chloride or an acyl chloride capable of an exchange reaction. Specific reagents and procedures used to carry out such activation reactions will be known to those of ordinary skill in the art and are described in the pertinent texts and literature.

Analogously, when a first portion of the suture surface is functionalized with sulfhydryl, the electrophilic groups present on a second portion of the suture surface may be groups that react with a sufthydryl moiety. Such reactive groups include those that form thioester linkages upon reaction with a sulfhydryl group, such as those described in PCT Publication No. WO 00/62827 to Wallace et al. As explained in detail therein, such “sulfhydryl reactive” groups include, but are not limited to: mixed anhydrides; ester derivatives of phosphorus; ester derivatives of p-nitrophenol, p-nitrothiophenol and pentafluorophenol; esters of substituted hydroxylamines, including N-hydroxyphthalimide esters, N-hydroxysuccinimide esters, N-hydroxysulfosuccinimide esters, and N-hydroxyglutarinide esters; esters of 1-hydroxybenzotriazole; 3-hydroxy-3,4-dihydro-benzotriazin-4-one; 3-hydroxy-3,4-dihydro-quinazoline-4-one; carbonylimidazole derivatives; acid chlorides; ketenes; and isocyanates. With these sulfhydryl reactive groups, auxiliary reagents can also be used to facilitate bond formation, e.g., 1-ethyl-[3-dimethylaminopropyl]carbodiimide can be used to facilitate coupling of sulfhydryl groups to carboxyl-containing groups.

In addition to the sulfhydryl reactive groups that form thioester linkages, various other sulfhydryl reactive functionalities can be utilized that form other types of linkages. For example, compounds that contain methyl imidate derivatives form imido-thioester linkages with sulthydryl groups. Alternatively, sulthydryl reactive groups can be employed that form disulfide bonds with sulthydryl groups, such groups generally have the structure —S—S—Ar where Ar is a substituted or unsubstituted nitrogen-containing heteroaromatic moiety or a non-heterocyclic aromatic group substituted with an electron-withdrawing moiety, such that Ar may be, for example, 4-pyridinyl, o-nitrophenyl, m-nitrophenyl, p-nitrophenyl, 2,4-dinitrophenyl, 2-nitro-4-benzoic acid, 2-nitro-4-pyridinyl, etc. In such instances, auxiliary reagents, e.g., mild oxidizing agents such as hydrogen peroxide can be used to facilitate disulfide bond formation.

Yet another class of sulfhydryl reactive groups form thioether bonds with sulfhydryl groups. Such groups include, inter alia, maleimido, substituted maleimido, haloalkyl, epoxy, imino, and aziridino, as well as olefins (including conjugated olefins) such as ethenesulfonyl, etheneimino, acrylate, methacrylate, and a,(3-unsaturated aldehydes and ketones.

When a first portion of the suture surface is functionalized with —OH, the electrophilic functional groups on the second portion of the suture surface must react with hydroxyl groups. The hydroxyl group may be activated as described above with respect to carboxylic acid groups, or it may react directly in the presence of base with a sufficiently reactive electrophile such as an epoxide group, an aziridine group, an acyl halide, or an anhydride.

When a first portion of the suture surface is functionalized with an organometallic nucleophile such as a Grignard functionality or an alkyllithium group, suitable electrophilic functional groups for reaction therewith are those containing carbonyl groups, including, by way of example, ketones and aldehydes.

It will also be appreciated that certain functional groups can react as nucleophiles or as electrophiles, depending on the selected reaction partner and/or the reaction conditions. For example, a carboxylic acid group can act as a nucleophile in the presence of a fairly strong base, but generally acts as an electrophile allowing nucleophilic attack at the carbonyl carbon and concomitant replacement of the hydroxyl group with the incoming nucleophile.

Table 1, below illustrates, solely by way of example, representative complementary pairs of electrophilic and nucleophilic functional groups that may be employed in functionalizing a first portion of the suture surface (e.g., R₁ in Table 1) and a second portion of the suture surface (e.g., R₂ in Table 1).

TABLE 1

| REPRESEN- | | |
|---|---|---|
| TATIVE | | |
| NUCLEO- | | |
| PHILIC | | |
| COMPO- | REPRESENTATIVE | |
| NENT | ELECTROPHILIC COMPONENT | |
| (A, FN_{NU}) | (B, FN_{EL}) | RESULTING LINKAGE |
| R¹—NH₂ | R²—O—(CO)—O—N(COCH₂) | R¹—NH—(CO)—O—R² |
| | (succinimidyl carbonate terminus) | |
| R¹—SH | R²—O—(CO)—O—N(COCH₂) | R¹—S—(CO)—O—R² |
| R¹—OH | R²—O—(CO)—O—N(COCH₂) | R¹—S—(CO)—R² |
| R¹—NH₂ | R²—O(CO)—CH═CH₂ | R¹—NH—CH₂CH₂—(CO)—O—R² |
| | (acrylate terminus) | |
| R¹—SH | R²—O—(CO)—CH═CH₂ | R¹—S—CH₂CH₂—(CO)—O—R² |
| R¹—OH | R²—O—(CO)—CH═CH₂ | R¹—O—CH₂CH₂—(CO)—O—R² |
| R¹—NH₂ | R²—O(CO)—(CH₂)₃—CO₂N(COCH₂) | R¹—NH—(CO)—(CH₂)₃—(CO)—OR² |
| | (succinimidyl glutarate terminus) | |
| R¹—SH | R²—O(CO)—(CH₂)₃—CO₂—N(COCH₂) | R¹—S—(CO)—(CH₂)₃—(CO)—OR² |
| R¹—OH | R²—O(CO)—(CH₂)₃—CO₂—N(COCH₂) | R¹—O—(CO)—(CH₂)₃—(CO)—OR² |
| R¹—NH₂ | R²—O—CH₂—CO₂—N(COCH₂) | R¹—NH—(CO)—CH₂—OR² |
| | (succinimidyl acetate terminus) | |
| R¹—SH | R²—O—CH₂—CO₂—N(COCH₂) | R¹—S—(CO)—CH₂—OR² |
| R¹—OH | R²—O—CH₂—CO₂—N(COCH₂) | R¹—O—(CO)—CH₂—OR² |
| R¹—NH₂ | R²—O—NH(CO)—(CH₂)₂—CO₂—N(COCH₂) | R¹—NH—(CO)—(CH₂)₂—(CO)—NH—OR² |
| | (succinimidyl succinamide terminus) | |
| R¹—SH | R²—O—NH(CO)—(CH₂)₂—CO₂—N(COCH₂) | R¹—S—(CO)—(CH₂)₂—(CO)—NH—OR² |
| R¹—OH | R²—O—NH(CO)—(CH₂)₂—CO₂—N(COCH₂) | R¹—O—(CO)—(CH₂)₂—(CO)—NH—OR² |
| R¹—NH₂ | R²—O—(CH₂)₂—CHO | R¹—NH—(CO)—(CH₂)₂—OR² |
| | (propionaldehyde terminus) | |
| R¹—NH₂ | 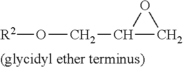 | R¹—NH—CH₂—CH(OH)—CH₂—OR² and R¹—N[CH₂—CH(OH)—CH₂—OR²]₂ |
| R¹—NH₂ | R²—O—(CH₂)₂—N═C═O | R¹—NH—(CO)—NH—CH₂—OR² |
| | (isocyanate terminus) | |
| R¹—NH₂ | R²—SO₂—CH═CH₂ | R¹—NH—CH₂CH₂—SO₂—R² |
| | (vinyl sulfone terminus) | |
| R¹—SH | R²—SO₂—CH═CH₂ | R¹—S—CH₂CH₂—SO₂—R² |

In embodiments, the first portion of the suture surface may be functionalized with a first click-reactive member and a second portion of the suture surface may be functionalized with a second click-reactive member complementary to the first click-reactive member. The “click-reactive members” are meant to include those reactive members used in the processes known to those skilled in the art as Click chemistry.

Click chemistry refers to a collection of reactive members having a high chemical potential energy capable of producing highly selective, high yield reactions. The reactive members react to form extremely reliable molecular connections in most solvents, including physiologic fluids, and often do not interfere with other reagents and reactions. Examples of click chemistry reactions include Huisgen cycloaddition, Diels-Alder reactions, thiol-alkene reactions, and maleimide-thiol reactions.

Huisgen cycloaddition is the reaction of a dipolarophile with a 1,3-dipolar compound that leads to 5-membered (hetero)cycles. Examples of dipolarophiles are alkenes and alkynes and molecules that possess related heteroatom functional groups (such as carbonyls and nitriles). 1,3-Dipolar compounds contain one or more heteroatoms and can be described as having at least one mesomeric structure that represents a charged dipole. They include nitril oxides, azides, and diazoalkanes. Metal catalyzed click chemistry is an extremely efficient variant of the Huisgen 1,3-dipolar cycloaddition reaction between alkyl-aryly-sulfonyl azides, C—N triple bonds and C—C triple bonds which is well-suited herein. The results of these reactions are 1,2 oxazoles, 1,2,3 triazoles or tetrazoles. For example, 1,2,3 triazoles are formed by a copper catalyzed Huisgen reaction between alkynes and alkly/aryl azides. Metal catalyzed Huisgen reactions proceed at ambient temperature, are not sensitive to solvents, i.e., nonpolar, polar, semipolar, and are highly tolerant of functional groups. Non-metal Huisgen reactions (also referred to as strain promoted cycloaddition) involving use of a substituted cyclooctyne, which possesses ring strain and electron-withdrawing substituents such as fluorine, that together promote a [3+2] dipolar cycloaddition with azides are especially well-suited for use herein due to low toxicity as compared to the metal catalyzed reactions. Examples include DIFO and DIMAC. Reaction of the alkynes and azides is very specific and essentially inert against the chemical environment of biological tissues. One reaction scheme may be represented as:

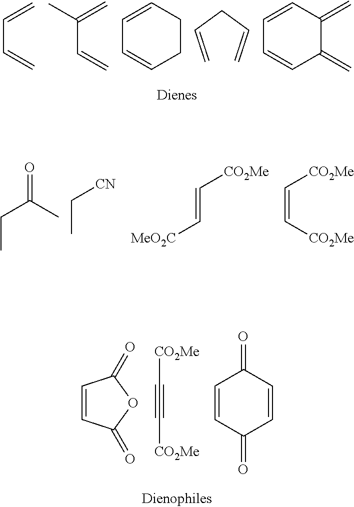

where R and R′ represent the first and second portions of the suture surface.

The Diels-Alder reaction combines a diene (a molecule with two alternating double bonds) and a dienophile (an alkene) to make rings and bicyclic compounds. Examples include:

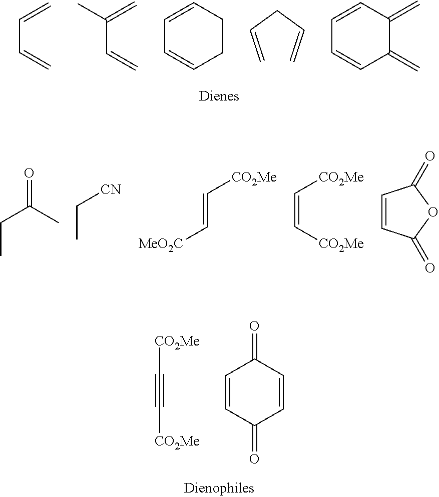

The thiol-alkene (thiol-ene) reaction is a hydrothiolation, i.e., addition of RS-H across a C═C bond. The thiol-ene reaction proceeds via a free-radical chain mechanism. Initiation occurs by radical formation upon UV excitation of a photoinitiator or the thiol itself Thiolene systems form ground state charge transfer complexes and therefore photopolymerize even in the absence of initiators in reasonable polymerization times. However, the addition of UV light increases the speed at which the reaction proceeds. The wavelength of the light can be modulated as needed, depending upon the size and nature of the constituents attached to the thiol or alkene. A general thiol-ene coupling reaction mechanism is represented below:

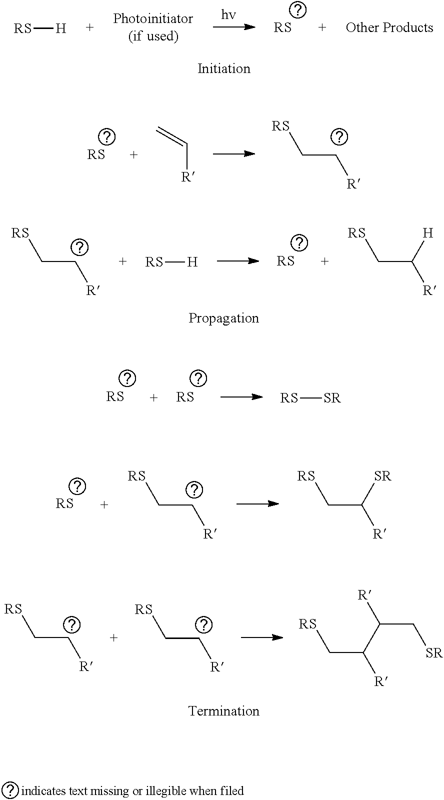

In embodiments, a first portion of the suture surface and a second portion of the suture surface are functionalized to include a first click-reactive member which is an alkyne and a second click-reactive member which is an azide, respectively. In embodiments, a first portion of the suture surface and a second portion of the suture surface are functionalized to include a first click-reactive member which includes an azide group and a second click-reactive member which is an alkyne, respectively. The first and second click-reactive members are intended to react and covalently bond the first and second portions of the suture surface at a physiologic pH. However, in some embodiments, the first and second click-reactive members may react quicker or more completely following the addition of a catalyst, such as a pH modifier, a metal ion catalyst or the introduction of heat or radiation. In embodiments, the addition of UV radiation may enhance the formation of a covalent bond between the first and second click-reactive members. In embodiments, the addition of a metal catalyst, e.g., transition metal ions such as copper ions may assist with the formation of a covalent bond between the first and second click-reactive members.

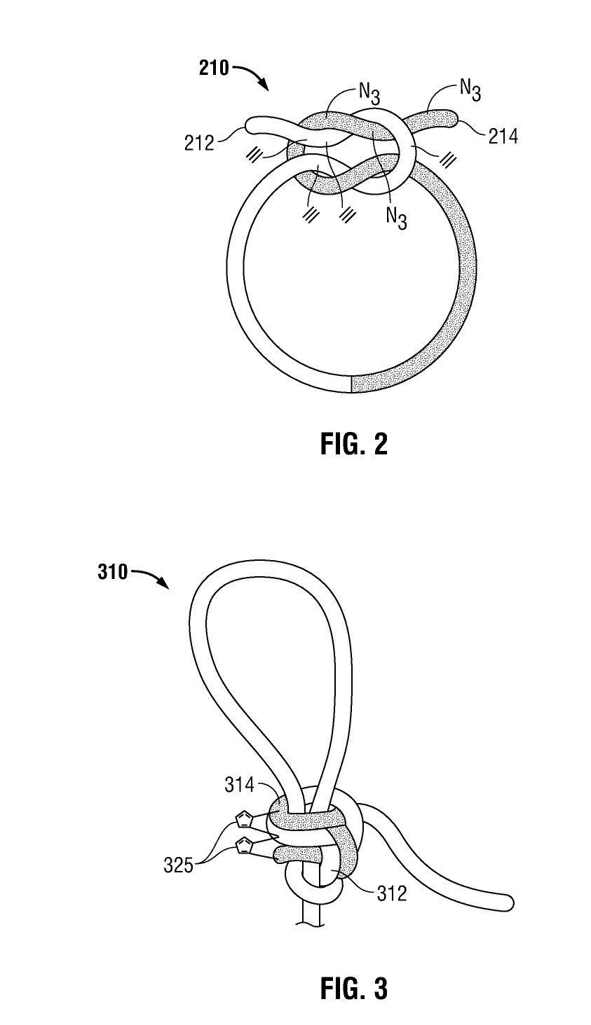

As schematically shown in FIG. 2, first surface portion 112 of suture 110 includes first reactive members, in this case azide groups and second portion 114 of suture 110 includes second reactive members, in this case alkyne groups. After the suture knot is formed, at least sections of the first and second portions of the suture surface are positioned next to each other and in close proximity to allow for the complimentary groups to react and form a chemical bond. As those skilled in the art will recognize, reaction times between the azide and alkyne members can be reduced from about 24 hours at room temperature to mere seconds at room temperature by the presence of transition metal ions, such as copper ions.

As schematically shown in FIG. 3, following interaction between the complimentary reactive members, knotted suture 310 includes sections wherein first portion 312 is covalently bound to sections of second portion 314 via triazole linkages 325. This chemical bond increases the knot strength and in the case of slidable knots may allow for a smaller profile knot.

The first and second reactive members may be positioned on the surface of bioabsorbable polymeric suture using any variety of suitable chemical processes. With respect to the first and second reactive members on the first and second portions of the suture surface, it is contemplated that a plurality of first and/or second reactive members may be present and may be terminally located, or alternatively located anywhere along the length of the polymer chain used to form the suture.

For example, the monomers from which the bioabsorbable polymeric suture is made can be functionalized so that the first and/or second reactive members appear along the length of the bioabsorbable polymer. In such embodiments, the monomers can be initially functionalized with a member such as a halogen to provide a reactive site at which the desired first reactive member can be attached after polymerization. Thus, for example, a cyclic lactone (e.g., glycolide, lactide, caprolactone, etc.) can be halogenated and then polymerized using known techniques for ring opening polymerization. Once polymerized, the halogenated sites along the resulting polyester chain can be functionalized with the first and/or second reactive members. For example, the halogenated polyester can be reacted with sodium azide to provide azide groups along the polymer chain and/or with propagyl alcohol to provide alkyne groups along the polymer chain. See, R. Riva et al., Polymer 49, pages 2023-2028 (2008) for a description of such reaction schemes. In another example, a propargyl group may be introduce into a cyclic carbonate monomer to form 5-methyl-5-propargyloxycarbonyl-1,3-dioxan-2-one (MPC) which is polymerizable with lactide to form p(LA-co-MPC). See, Q. Shi et al., Biomaterials, 29, pages 1118-1126 (2008). Alternatively, a preformed biodegradable polyester can be halogenated by reaction with a non-nucleophilic strong base, such as lithium diisopropylamide, followed by electrophilic substitution with iodine chloride. The halogenated polyester is then reacted with sodium azide and/or propagyl alcohol to provide azide and/or alkyne groups, respectively. Other methods for functionalizing lactones are described in Jerome et al., Advanced Drug Delivery Reviews, 60, pages 1056-1076 (2008). The entire disclosure of each of these articles is incorporated herein by this reference.

In other embodiments, the bioabsorbable polymeric suture is functionalized after it has been fabricated into the desired form. For example, bioabsorbable polymeric fibers can be functionalized after the extrusion or spinning process. In embodiments, the fibers are surface treated and then activated with the first reactive member (optionally with a coupling agent (e.g., a silane coupling agent) being used). Surface activation of bioabsorbable and biocompatible aliphatic polyesters can be achieved by acid or base hydrolysis, treatment by means of cold plasma, by chemical reactions or electromagnetic radiations. It is contemplated that such surface activation can be performed before or after the fibers are made into a multifilament structure.

Hydrolysis can be conducted in the presence of an aqueous solution of a base or an acid to accelerate surface reaction, inasmuch as excessively long processes of activation can induce a reduction in molecular weight and thus in the mechanical properties of the material. Suitable bases for obtaining watery solutions suited to the aim are, for example, strong alkalis, such as LiOH, Ba(OH)₂, Mg(OH)₂, NaOH, KOH, Na₂CO₃, Ca(OH)₂ and the weak bases, such as for example NH₄ OH and the ammines such as methylamine, ethylamine, diethylamine and dimethylamine. Acids suitable for surface hydrolysis treatments can be chosen, for example, from among HCl, HClO₃, HClO₄, H₂ SO₃, H₂ SO₄, H₃ PO₃, H₃ PO₄, HI, HIO₃, HBr, lactic acid, glycolic acid. Surface activation by means of hydrolysis can be conducted at temperatures preferably comprised between 0 degrees Celsius and the material softening temperature. Plasma treatment can be carried out both in the presence of a reactive gas, for example air, Ar, O₂ with the formation of surface activation of oxygenate type, such as —OH, —CHO, —COOH.

Surface treatment, whether hydrolytic or with plasma, can remain unaltered or can be followed by further chemical modifications to provide the first and/or second reactive members on the bioabsorbable polymeric suture. Thus, for example, the COONa members generated by a base hydrolysis can be subsequently converted into COOH members by treatment with strong mineral acids. Further, the surface freeing of alcoholic members by means of a hydrolysis process can be followed by reaction by means of the addition of a compound provided with functional group or groups able to react with surface alcoholic groups, such as for example by means of the addition of an anhydride such as succinic anhydride, with the conversion of —OH groups into —O—CO—CH₂—CH₂—COOH groups. Suitable surface activation techniques are disclosed in U.S. Pat. No. 6,107,453, the entire disclosure of which is incorporated herein by this reference.

In embodiments, a plurality of different first and second reactive members may be positioned on each of the first and second portions of the suture surface.

In embodiments, a chemical knot is formed by a process which includes providing a surgical suture which includes a first portion having at least one first reactive member and a second portion having at least one second complimentary reactive member. The first portion of the surgical suture is positioned in close proximity to the second portion of the suture. The first portion of the suture is allowed to bond to the second portion of the suture via the first and second complimentary reactive groups to form a chemical knot. In embodiments, a catalyst may be utilized.

In embodiments, the first and second portions of the suture surface may be positioned in close proximity without forming a physical suture knot. In embodiments, the first and second portions of the suture surface may be positioned in close proximity after forming a physical suture knot. Any knot formation suitable for medical use including slidable and non-slidable knots may be formed. Some non-limiting examples include Duncan's Loop, Nicky's Knot, Tennessee Sliders, Roeders Knot, SMC knots, and Weston Knots.

In some embodiments, at least one bioactive agent may be combined with the sutures described herein. For example, a bioactive agent may be combined with the polymer used to form the suture, and/or a bioactive agent may be coated onto any portion of the suture including the first, second and/or spacer portions described herein. The at least one agent may be freely released by the suture or may be chemically bound to the surface of the suture

The term “bioactive agent”, as used herein, is used in its broadest sense and includes any substance or mixture of substances that have clinical use. Consequently, bioactive agents may or may not have pharmacological activity per se, e.g., a dye, or fragrance. Alternatively a bioactive agent could be any agent which provides a therapeutic or prophylactic effect, a compound that affects or participates in tissue growth, cell growth, cell differentiation, an anti-adhesive compound, a compound that may be able to invoke a biological action such as an immune response, a compound that adds lubricity to the outer surface of the suture, or could play any other role in one or more biological processes. It is envisioned that the bioactive agent may be applied to the present suture materials in any suitable form of matter, e.g., films, powders, liquids, gels and the like.

Examples of classes of bioactive agents which may be utilized in accordance with the present disclosure include anti-adhesives, antimicrobials, analgesics, antipyretics, anesthetics, antiepileptics, antihistamines, anti-inflammatories, cardiovascular drugs, diagnostic agents, sympathomimetics, cholinomimetics, antimuscarinics, antispasmodics, hormones, growth factors, muscle relaxants, adrenergic neuron blockers, antineoplastics, immunogenic agents, immunosuppressants, gastrointestinal drugs, diuretics, steroids, lipids, lipopolysaccharides, polysaccharides, lubricants, oils, and enzymes. It is also intended that combinations of bioactive agents may be used.

Suitable antimicrobial agents which may be included as a bioactive agent in the suture materials of the present disclosure include triclosan, also known as 2,4,4′-trichloro-2′-hydroxydiphenyl ether, chlorhexidine and its salts, including chlorhexidine acetate, chlorhexidine gluconate, chlorhexidine hydrochloride, and chlorhexidine sulfate, silver and its salts, including silver acetate, silver benzoate, silver carbonate, silver citrate, silver iodate, silver iodide, silver lactate, silver laurate, silver nitrate, silver oxide, silver palmitate, silver protein, and silver sulfadiazine, polymyxin, tetracycline, aminoglycosides, such as tobramycin and gentamicin, rifampicin, bacitracin, neomycin, chloramphenicol, miconazole, quinolones such as oxolinic acid, norfloxacin, nalidixic acid, pefloxacin, enoxacin and ciprofloxacin, penicillins such as oxacillin and pipracil, nonoxynol 9, fusidic acid, cephalosporins, and combinations thereof. In addition, antimicrobial proteins and peptides such as bovine lactoferrin and lactoferricin B and antimicrobial polysaccharides such as fucans and derivatives may be included as a bioactive agent in the sutures of the present disclosure.

Other bioactive agents which may be included as a bioactive agent in the sutures in accordance with the present disclosure include: local anesthetics; non-steroidal antifertility agents; parasympathomimetic agents; psychotherapeutic agents; tranquilizers; decongestants; sedative hypnotics; steroids; sulfonamides; sympathomimetic agents; vaccines; vitamins; antimalarials; anti-migraine agents; anti-parkinson agents such as L-dopa; anti-spasmodics; anticholinergic agents (e.g. oxybutynin); antitussives; bronchodilators; cardiovascular agents such as coronary vasodilators and nitroglycerin; alkaloids; analgesics; narcotics such as codeine, dihydrocodeinone, meperidine, morphine and the like; non-narcotics such as salicylates, aspirin, acetaminophen, d-propoxyphene and the like; opioid receptor antagonists, such as naltrexone and naloxone; anti-cancer agents; anti-convulsants; anti-emetics; antihistamines; anti-inflammatory agents such as hormonal agents, hydrocortisone, prednisolone, prednisone, non-hormonal agents, allopurinol, indomethacin, phenylbutazone and the like; prostaglandins and cytotoxic drugs; estrogens; antibacterials; antibiotics; anti-fungals; anti-virals; anticoagulants; anticonvulsants; antidepressants; antihistamines; and immunological agents.

Other examples of suitable bioactive agents which may be included in the present suture materials include viruses and cells, peptides, polypeptides and proteins, analogs, muteins, and active fragments thereof, such as immunoglobulins, antibodies, cytokines (e.g. lymphokines, monokines, chemokines), blood clotting factors, hemopoietic factors, interleukins (IL-2, IL-3, IL-4, IL-6), interferons ((3-IFN, (a-IFN and y-IFN), erythropoietin, nucleases, tumor necrosis factor, colony stimulating factors (e.g., GCSF, GM-CSF, MCSF), insulin, anti-tumor agents and tumor suppressors, blood proteins, gonadotropins (e.g., FSH, LH, CG, etc.), hormones and hormone analogs (e.g., growth hormone), vaccines (e.g., tumoral, bacterial and viral antigens); somatostatin; antigens; blood coagulation factors; growth factors (e.g., nerve growth factor, insulin-like growth factor); protein inhibitors, protein antagonists, and protein agonists; nucleic acids, such as antisense molecules, DNA and RNA; oligonucleotides; polynucleotides; and ribozymes.

In still other embodiments, the sutures described herein may include an additional lubricant material. The addition of a lubricant material such as mineral oil may make the suture more slippery thereby decreasing tissue drag during the closing of a wound. Since the sutures described herein are capable of forming both a physical knot and a chemical knot, the suture knot formed from a lubricated suture is less likely to slip or become unraveled due to the chemical bond between portions of the suture. Although any material suitable for lubricating an implantable surgical suture may be applied to the suture, in some embodiments, the lubricant may be an oil or a hydrophilic coating. Some examples of useful lubricants can be found in: U.S. Pat. Nos. 5,041,100, 4,976,703, 4,961,954, 4,835,003, 4,801,475, 4,743,673, 4,729,914, 4,666,437, 4,589,873, 4,585,666, 4,487,808, 4,373,009, 4,100,309, 4,459,317, 4,487,808, and 4,729,914 each of which are incorporated herein by reference. Other aspects of the invention are defined in the following clauses:

Clause 1. A surgical suture capable of forming a chemical knot comprising

a suture comprising at least one filament,

a first portion of the filament having at least one first reactive member thereon; and

a second portion of the filament having at least one second complimentary reactive member thereon;

wherein the first and second reactive members are capable of chemically bonding the first portion of the filament to the second portion of the filament.

Clause 2. The surgical suture of clause 1 wherein the suture comprises a single filament.

Clause 3. The surgical suture of clause 1 wherein the suture comprises a plurality of filaments.

Clause 4. The surgical suture of any one of clauses 1-3 wherein the first and second complimentary reactive members are selected from the group consisting of electrophilic groups, nucleophilic groups and combinations thereof.

Clause 5. The surgical suture of any one of clauses 1-3 wherein the first and second complimentary reactive members are selected from the group consisting of alkynes, azides, and combinations thereof.

Clause 6. The surgical suture of any one of clauses 1-5 wherein the first portion of the filament represents about one-half a length of the surgical suture.

Clause 7. The surgical suture of any one of clauses 1-5 wherein the first and second portions of the filament comprise a candy-cane pattern.

Clause 8. The surgical suture of any one of clauses 1-7 further comprising at least one bioactive agent.

Clause 9. The surgical suture of clause 8 wherein the at least one bioactive agent is a lubricant.

Clause 10. The surgical suture of any one of clauses 1-9 further comprising a spacer portion.

Clause 11. A method comprising: positioning a first portion of a suture in close proximity to the second portion of the suture, the first portion comprising a first reactive member and a second portion comprising a second complimentary reactive member; and bonding the first portion of the suture to the second portion of the suture via the first and second complimentary reactive groups to form a chemical knot.

Clause 12. The method of clause 11 wherein the surgical suture is a monofilament fiber.

Clause 13. The method of clause 11 wherein the surgical suture is a multifilament fiber.

Clause 14. The method of any one of clauses 11-13 wherein the first and second complimentary reactive members are selected from the group consisting of electrophilic groups, nucleophilic groups and combinations thereof.

Clause 15. The method of any one of clauses 11-13 wherein the first and second complimentary reactive members are selected from the group consisting of alkynes, azides, and combinations thereof.

Clause 16. The method of any one of clauses 11-15 wherein the first portion of the suture represents about one-half a length of the surgical suture.

It will be understood that various modifications may be made to the embodiments disclosed herein. Therefore, the above description should not be construed as limiting, but merely as exemplifications within the scope and spirit of the claims appended hereto.

What is claimed is:

1. A surgical suture capable of forming a chemical knot comprising a suture comprising at least one filament, a first portion of the filament having at least one first reactive member thereon; and
a second portion of the filament having at least one second complimentary reactive member thereon;
wherein the first and second reactive members are capable of chemically bonding the first portion of the filament to the second portion of the filament.

2. The surgical suture of claim 1 wherein the suture comprises a single filament.

3. The surgical suture of claim 1 wherein the suture comprises a plurality of filaments.

4. The surgical suture of claim 1 wherein the first and second complimentary reactive members are selected from the group consisting of electrophilic groups, nucleophilic groups and combinations thereof.

5. The surgical suture of claim 1 wherein the first and second complimentary reactive members are selected from the group consisting of alkynes, azides, and combinations thereof.

6. The surgical suture of claim 1 wherein the first portion of the filament represents about one-half a length of the surgical suture.

7. The surgical suture of claiml wherein the first and second portions of the filament comprise a candy-cane pattern.

8. The surgical suture of claim 1 further comprising at least one bioactive agent.

9. The surgical suture of claim 8 wherein the at least one bioactive agent is a lubricant.

10. The surgical suture of claim 1 further comprising a spacer portion.

11. A method comprising:
positioning a first portion of a suture in close proximity to the second portion of the suture, the first portion comprising a first reactive member and a second portion comprising a second complimentary reactive member; and
bonding the first portion of the suture to the second portion of the suture via the first and second complimentary reactive groups to form a chemical knot.

12. The method of claim 11 wherein the surgical suture is a monofilament fiber.

13. The method of claim 11 wherein the surgical suture is a multifilament fiber.

14. The method of claim 11 wherein the first and second complimentary reactive members are selected from the group consisting of electrophilic groups, nucleophilic groups and combinations thereof.

15. The method of claim 11 wherein the first and second complimentary reactive members are selected from the group consisting of alkynes, azides, and combinations thereof.

16. The method of claim 11 wherein the first portion of the suture represents about one-half a length of the surgical suture.
